(19) United States
(12) Patent Application Publication
(10) Pub. No.: US 2026/0041903 A1
(43) Pub. Date: Feb. 12, 2026

(54) Stone Therapy Device

(71) Applicant: Maria Scarola Bell, Toms River, NJ (US)

(72) Inventor: Maria Scarola Bell, Toms River, NJ (US)

(21) Appl. No.: 19/292,121

(22) Filed: Aug. 6, 2025

Related U.S. Application Data
(60) Provisional application No. 63/679,653, filed on Aug. 6, 2024.

(51) Int. Cl. A61N 1/02

(52) U.S. Cl. CPC A61N 1/02

(57) ABSTRACT

A stone therapy device is disclosed, which is an innovative set of devices that revolutionizes traditional stone therapy for skin rejuvenation. The stone therapy device comprises specially designed face forms crafted from genuine tiger's eye gemstone. The face forms are sized and shaped to target key areas, such as the forehead, under-eye region, smile lines, and the sides of the mouth and nose. The stone therapy device uniquely combines the timeless properties of gemstones with advanced technology, creating a groundbreaking approach to skincare. By harnessing the alleged energetic qualities of tiger's eye in conjunction with modern piezoelectric technology, the device aims to deliver enhanced wellness benefits in a previously unexplored therapeutic context.

### CROSS-REFERENCE TO RELATED APPLICATION

The present application claims priority to, and the benefit of, U.S. Provisional Application No. 63/679,653, which was filed on Aug. 6, 2024, and is incorporated herein by reference in its entirety.

### FIELD OF THE INVENTION

The present invention relates generally to the field of stone therapy devices. More specifically, the present invention relates to a facial patch for rejuvenating the skin. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in stone therapy devices. Generally, people with visible lines and wrinkles or puffy skin may be looking for products to help alleviate these issues. Further, dull complexion and uneven skin tone can be frustrating to deal with. Additionally, people may suffer from low energy, lack of motivation, stress, tension, and difficulty concentrating without a way to relieve any of these ailments.

Aging of the face is known to result in reduced elasticity of the skin leaving wrinkles on the skin. It is known that a person can have some control over the acceleration of the wrinkle effect by maintaining good nutrition and exercise habits. Improvements can also be gained by application of various novel cosmetic skin care products and even cosmetic face lift surgeries, but those may not be cost effective solutions for all categories of people out there.

Accordingly, there is a demand for an improved stone therapy device that utilizes genuine tiger's eye gemstone to construct facial patches which can help diminish wrinkles, reduce puffiness, and soothe sensitive skin. More particularly, there is a demand for a stone therapy device that features patches that can be applied around the forehead, under the eye, and on dimple lines to revitalize the skin.

Therefore, there exists a long felt need in the art for a stone therapy device that provides users with stone therapy for rejuvenating the skin. There is also a long felt need in the art for a stone therapy device that features patches that can be applied around the forehead, under the eye, and on dimple lines to revitalize the skin. Further, there is a long felt need in the art for a stone therapy device that utilizes genuine tiger's eye gemstones to construct the patches which can help diminish wrinkles, reduce puffiness, and soothe sensitive skin. Moreover, there is a long felt need in the art for a device that potentially helps boost energy and motivation while balancing the endocrine system. Further, there is a long felt need in the art for a stone therapy device that offers a method of alternative medicine that provides a holistic approach to skin care and wellbeing. Finally, there is a long felt need in the art for a stone therapy device that harnesses the alleged energetic qualities of tiger's eye in conjunction with modern piezoelectric technology.

The subject matter disclosed and claimed herein, in one embodiment thereof, comprises a stone therapy device. The device is an innovative set of devices that revolutionizes traditional stone therapy for skin rejuvenation. The stone therapy device comprises specially designed face forms crafted from genuine tiger's eye gemstone. The face forms are sized and shaped to target key areas, such as the forehead, under-eye region, smile lines, and the sides of the mouth and nose. The stone therapy device uniquely combines the timeless properties of gemstones with advanced technology, creating a groundbreaking approach to skincare. By harnessing the alleged energetic qualities of tiger's eye in conjunction with modern piezoelectric technology, the device aims to deliver enhanced wellness benefits in a previously unexplored therapeutic context.

In this manner, the stone therapy device of the present invention accomplishes all of the forgoing objectives and provides users with patches that can be applied around a user's face to revitalize the skin. The device utilizes genuine tiger's eye gemstone to construct the patches. The device provides a holistic approach to skincare.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a stone therapy device. The stone therapy device comprises face forms/patches crafted from genuine tiger's eye gemstone. The stone therapy device uniquely combines the timeless properties of gemstones with advanced piezoelectric technology, creating a groundbreaking approach to skincare. The device delivers enhanced wellness benefits in a previously unexplored therapeutic context.

In one embodiment, the stone therapy device provides a holistic approach to skincare and wellbeing. The device provides users with various facial patches for rejuvenating the skin. The unique composition of the facial patches potentially helps boost energy and motivation while balancing the endocrine system.

In one embodiment, the stone therapy device comprises a body component configured as a facial patch to be applied to various areas of a user's face, during use. The body component is configured in various shapes and sizes to correspond with various areas of a user's face. The body component comprises a top surface facing out from a user's face, a bottom surface that contacts a user's face, and a curved or rounded side edge, which defines the shape of the patch.

In one embodiment, the body component is configured in a curved, teardrop-like shape, which is sized and shaped to be positioned under a user's cheekbone, under a user's eye area and/or along the sides of a user's nose. In another embodiment, the body component is configured in a curved, oblong oval shape with a protruding bulbous area extending from the middle edge of the patch. This embodiment is sized and shaped to be positioned on a user's forehead and above a user's eyes, with the protruding bulbous area being positioned in the flat area between a user's eyebrows. In another embodiment, the body component is configured in a curved, oblong oval shape, which is sized and shaped to be positioned above or below a user's mouth and extends around the sides of a user's mouth. The scope of invention should not be limited to the various exemplary shapes and sizes. There can be many other possible shapes with different sizes that can be derived based on users need.

In one embodiment, the shape of the body component is designed to not only fit comfortably on the designated area of the face, but to also taper at the edges to minimize creasing and pressure on the skin, as not to create additional wrinkles. Further, the body component can be specially designed and custom matched to the facial contours of a user's forehead, under eye region, smile lines, dimple lines, and the sides of the mouth and nose, etc. Thus, the stone therapy device ensures perfect fit for each facial zone, while preserving the natural properties of the gemstone. In use, the tiger's eye gemstone can help revitalize skin, by diminishing wrinkles, reducing puffiness, and soothing sensitive skin, as well as can help fortify blood, alleviate sluggishness, and bring back feelings of motivation, etc.

In one embodiment, the body component comprises an adhesive backing for adhering the patch to skin. However, the body component does not need an adhesive and there is unlimited use of the device without an adhesive. The adhesive backing comprises any suitable adhesive as is known in the art. Typically, the adhesive backing is hypoallergenic, latex-free, and suitable for daily usage. One suitable adhesive is acrylate of the kind designed for medical and surgical uses, although other adhesives may also be used. For example, the adhesive backing can be a hydrocolloid, a hydrogel, or a collagen, etc. The adhesive may have an applied thickness of approximately 1 to 2 mils. Further, the adhesive backing has an adhesion value that allows for general case of application and removal from skin. A preferred operable adhesion value is between 18 to 24 ounces/inch width, and more preferably about 21 ounces/inch width, so that the stone therapy device has a comfortable medium adhesion to facial skin. Typically, the stone therapy device can remain on skin for as little or long as desired up to 24 hours at a time and can be easily removed from skin by pulling the patch across itself 180° and peeling it off for disposal. Although as explained herein, the patch is preferably used one time, however, it should be understood that the patch can be used as many times as possible, until it loses its stickiness or wears off.

In one embodiment, the body component comprises a thin release liner, which forms a backing for the adhesive. In use, the release layer is grasped between a thumb and forefinger and completely pulled away from the adhesive backing. Once the release liner is removed, the adhesive backing of the body component is exposed for use. The release liner may be poly-coated craft paper with a silicone layer providing release capability for the adhesive backing of the body component. In use, the stone therapy devices are peeled away from the release liner and affixed to a user's face. In one embodiment, the stone therapy devices are delivered as a sheet of multiple patches die-cut from and releasably adhered to the release liner.

In one embodiment, the body component can be composed of all viable crystal types, as is known in the art.

In one embodiment, the body component is entirely composed of genuine tiger's eye gemstone. Utilizing genuine tiger's eye gemstones to construct the patches, can help diminish wrinkles, reduce puffiness, and soothe sensitive skin. Generally, tiger's eye gemstone is a golden-yellow to reddish-brown quartz gemstone. It is known for its unique optical phenomenon called chatoyancy. Tiger's eye exhibits chatoyancy due to the intergrowth of nearly parallel quartz crystals and altered crocidolite fibers. Tiger's eye is composed primarily of silicon dioxide (SiO2). As members of the quartz group, tiger's eye and the related blue-colored mineral hawk's eye gain their silky, lustrous appearance from the parallel intergrowth of quartz crystals and altered amphibole fibers that have mostly turned into limonite. Tiger's eye is believed to offer many healing properties and benefits in your physical body as well as emotional benefits. Beyond your emotions, tiger's eye is thought to promote balance within your circulatory system and endocrine system. Some healers say that it fortifies the blood, giving you greater overall vitality and strength. The fortified blood is free to spread throughout your body, bringing greater balance as a whole. As for the endocrine system, some say tiger's eye brings your hormone production down to a normal level, helping your body level itself out. Further, biochemistry stabilizes, which is thought to help with everything from fertility to high blood pressure. According to some healers, it can even resolve issues with the gastrointestinal system while aligning the spine. Those who suffer from debilitating emotional issues, such as depression or seasonal affective disorder, may also benefit from the healing it brings. The sun-soaked stone has the potential to provide warmth on emotionally cold days. Thus, the application of the stone therapy device provides a holistic approach to skincare and potentially helps boost energy and motivation, while balancing the endocrine system. In use, the tiger's eye gemstone applied to the body component, can help fortify blood, alleviate sluggishness, and bring back feelings of motivation, etc.

In one embodiment, the body component also utilizes modern piezoelectric technology. Piezoelectric technology aims to deliver enhanced wellness benefits in a previously unexplored therapeutic context. Specifically, piezoelectric technology allows for the generation of electric fields (EFs) based on pressure, which modulate skin cell behaviors. Thus, the piezoelectric effect is the direct interaction between the mechanical and electrical states in crystalline materials, such as tiger's eye. Accordingly, the stone therapy device uniquely combines the timeless properties of gemstones with advanced piezoelectric technology, creating a groundbreaking approach to skincare.

Although the stone therapy device of the present invention is mainly developed for face, neck and décolleté areas (i.e., used as a face patch), it may also be used for application to other regions of the body as well.

In yet another embodiment, the stone therapy device comprises a plurality of indicia.

In yet another embodiment, a method of rejuvenating the face with facial patches is disclosed. The method includes the steps of providing a stone therapy device comprising a body component shaped in a face form. The method also comprises choosing a specifically sized body component which corresponds with a specific area of the face. Further, the method comprises removing the release liner from the adhesive backing of the body component. The method also comprises applying the body component to a specific area of the face. Finally, the method comprises removing the body component after a period of time and disposing it.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the stone therapy device of the present invention in accordance with the disclosed architecture;

FIG. 2 illustrates a perspective view of one embodiment of the stone therapy device of the present invention showing the device as an eye-shaped crystal patch in accordance with the disclosed architecture;

FIG. 3 illustrates a perspective view of one embodiment of the stone therapy device of the present invention showing the device as a forehead-shaped crystal patch in accordance with the disclosed architecture;

FIG. 4 illustrates a perspective view of one embodiment of the stone therapy device of the present invention showing the device as an around the mouth-shaped crystal patch in accordance with the disclosed architecture;

FIG. 5 illustrates a perspective view of one embodiment of the stone therapy device of the present invention showing the device in different styles and sizes in accordance with the disclosed architecture; and

FIG. 6 illustrates a flowchart showing the method of rejuvenating the face with facial patches in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a stone therapy device that provides users with stone therapy for rejuvenating the skin. There is also a long felt need in the art for a stone therapy device that features patches that can be applied around the forehead, under the eye, and on dimple lines to revitalize the skin. Further, there is a long felt need in the art for a stone therapy device that utilizes genuine tiger's eye gemstones to construct the patches which can help diminish wrinkles, reduce puffiness, and soothe sensitive skin. Moreover, there is a long felt need in the art for a device that potentially helps boost energy and motivation while balancing the endocrine system. Further, there is a long felt need in the art for a stone therapy device that offers a method of alternative medicine that provides a holistic approach to skin care and wellbeing. Finally, there is a long felt need in the art for a stone therapy device that harnesses the alleged energetic qualities of tiger's eye in conjunction with modern piezoelectric technology.

The present invention, in one exemplary embodiment, is a novel stone therapy device. The stone therapy device comprises specially designed face forms crafted from genuine tiger's eye gemstone. The face forms are sized and shaped to target key areas, such as the forehead, under-eye region, smile lines, and the sides of the mouth and nose. The stone therapy device uniquely combines the timeless properties of gemstones with advanced technology, creating a groundbreaking approach to skincare. By harnessing the alleged energetic qualities of tiger's eye in conjunction with modern piezoelectric technology, the device aims to deliver enhanced wellness benefits in a previously unexplored therapeutic context. The present invention also includes a novel method of rejuvenating the face with facial patches. The method includes the steps of providing a stone therapy device comprising a body component shaped in a face form. The method also comprises choosing a specifically sized body component which corresponds with a specific area of the face. Further, the method comprises removing the release liner from the adhesive backing of the body component. The method also comprises applying the body component to a specific area of the face. Finally, the method comprises removing the body component after a period of time and disposing it.

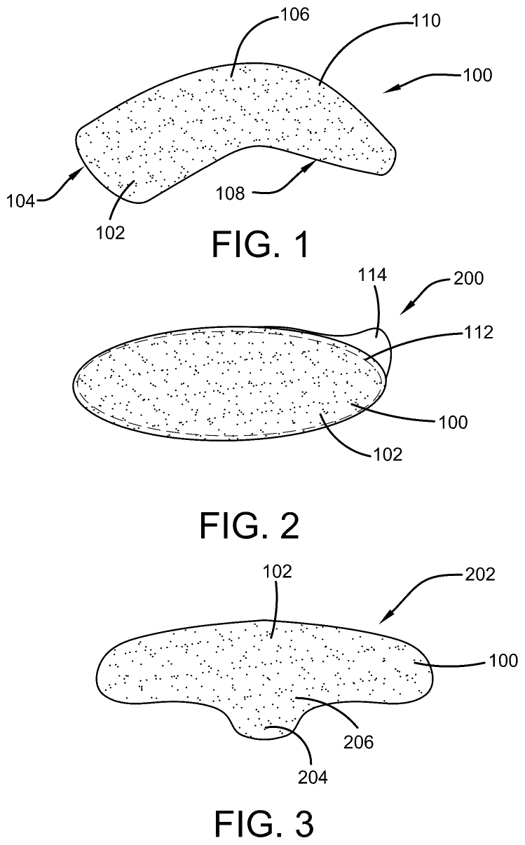

Referring initially to the drawings, FIG. 1 illustrates a perspective view of one embodiment of the stone therapy device 100 of the present invention. In the present embodiment, the stone therapy device 100 is an improved stone therapy device 100 that provides a user with facial forms/patches to deliver wellness benefits to a user's face. Specifically, the stone therapy device 100 comprises a body component 102 shaped in various facial forms and crafted of genuine tiger's eye gemstone 104. The stone therapy device 100 uniquely combines the timeless properties of gemstones with advanced piezoelectric technology, creating a groundbreaking approach to skincare. The device 100 delivers enhanced wellness benefits in a previously unexplored therapeutic context.

Generally, the stone therapy device 100 provides a holistic approach to skincare and wellbeing. The device 100 provides users with various facial patches (i.e., body component 102) for rejuvenating the skin. The unique composition of the facial patches 102 potentially helps boost energy and motivation while balancing the endocrine system.

Further, the stone therapy device 100 comprises a body component 102 configured as a facial patch to be applied to various areas of a user's face, during use. The body component 102 is configured in various shapes and sizes to correspond with various areas of a user's face. The body component 102 comprises a top surface 106 facing out from a user's face, a bottom surface 108 that contacts a user's face, and a curved or rounded side edge 110, which defines the shape of the patch (i.e., body component 102).

Additionally, the body component 102 comprises an adhesive backing 112 for adhering the patch to skin. However, the body component 102 does not need an adhesive backing 112 and has unlimited use without an adhesive. The adhesive backing 112 comprises any suitable adhesive as is known in the art. Typically, the adhesive backing 112 is hypoallergenic, latex-free, and suitable for daily usage. One suitable adhesive is acrylate of the kind designed for medical and surgical uses, although other adhesives may also be used. For example, the adhesive backing 112 can be a hydrocolloid, a hydrogel, or a collagen, etc. The adhesive may have an applied thickness of approximately 1 to 2 mils.

Further, the adhesive backing 112 has an adhesion value that allows for general case of application and removal from skin. A preferred operable adhesion value is between 18 to 24 ounces/inch width, and more preferably about 21 ounces/inch width, so that the stone therapy device 100 has a comfortable medium adhesion to facial skin. Typically, the stone therapy device 100 can remain on skin for as little or long as desired up to 24 hours at a time and can be easily removed from skin by pulling the patch 102 across itself 180° and peeling it off for disposal. Although as explained herein, the patch 102 is preferably used one time, however, it should be understood that the patch 102 can be used as many times as possible, until it loses its stickiness or wears off.

Furthermore, the body component 102 comprises a thin release liner 114, which forms a backing for the adhesive 112. In use, the release layer 114 is grasped between a thumb and forefinger and completely pulled away from the adhesive backing 112. Once the release liner 114 is removed, the adhesive backing 112 of the body component 102 is exposed for use. The release liner 114 may be poly-coated craft paper with a silicone layer providing release capability for the adhesive backing 112 of the body component 102. In use, the stone therapy devices 100 are peeled away from the release liner 114 and affixed to a user's face. In one embodiment, the stone therapy devices 100 are delivered as a sheet of multiple patches 102 die-cut from and releasably adhered to the release liner 114.

Typically, the body component 102 is entirely composed of genuine tiger's eye gemstone 104, but can be composed of other viable crystal types, as is known in the art. However, utilizing genuine tiger's eye gemstones 104 to construct the patches (i.e., body component 102), can help diminish wrinkles, reduce puffiness, and soothe sensitive skin. Generally, tiger's eye gemstone 104 is a golden-yellow to reddish-brown quartz gemstone. It is known for its unique optical phenomenon called chatoyancy. Tiger's eye 104 exhibits chatoyancy due to the intergrowth of nearly parallel quartz crystals and altered crocidolite fibers. Tiger's eye 104 is composed primarily of silicon dioxide (SiO2). As members of the quartz group, tiger's eye 104 and the related blue-colored mineral hawk's eye gain their silky, lustrous appearance from the parallel intergrowth of quartz crystals and altered amphibole fibers that have mostly turned into limonite.

Tiger's eye 104 is believed to offer many healing properties and benefits. It is thought to promote balance within your circulatory system and endocrine system. Some healers say that it fortifies the blood, giving you greater overall vitality and strength. The fortified blood is free to spread throughout your body, bringing greater balance as a whole. As for the endocrine system, some say tiger's eye 104 brings your hormone production down to a normal level, helping your body level itself out. Further, biochemistry stabilizes, which is thought to help with everything from fertility to high blood pressure. According to some healers, it can even resolve issues with the gastrointestinal system while aligning the spine. Those who suffer from debilitating emotional issues, such as depression or seasonal affective disorder, may also benefit from the healing it brings. The sun-soaked stone has the potential to provide warmth on emotionally cold days. Thus, the application of the stone therapy device 100 provides a holistic approach to skincare and potentially helps boost energy and motivation, while balancing the endocrine system. In use, the tiger's eye gemstone 104 applied to the body component 102, can help fortify blood, alleviate sluggishness, and bring back feelings of motivation, etc.

Additionally, in one embodiment, the body component 102 also utilizes modern piezoelectric technology. Piezoelectric technology aims to deliver enhanced wellness benefits in a previously unexplored therapeutic context. Specifically, piezoelectric technology allows for the generation of electric fields (EFs) based on pressure, which modulate skin cell behaviors. Thus, the piezoelectric effect is the direct interaction between the mechanical and electrical states in crystalline materials, such as tiger's eye 104. Accordingly, the stone therapy device 100 uniquely combines the timeless properties of gemstones with advanced piezoelectric technology, creating a groundbreaking approach to skincare.

Specifically, the stone therapy device 100 is comprised of specialized facial treatment forms designed for targeted application to specific areas of the face, including the forehead, under-eye region, cheeks, and neck. These forms are uniquely shaped and sized to conform to the anatomical contours of each facial zone, thereby ensuring optimal contact and treatment effectiveness.

The forms feature a distinctive design with strategically positioned ridges 201 and contoured surfaces that work to lift and support the skin when applied. The specific geometric configuration of these ridges 201 creates a mechanical lifting action that addresses skin concerns in each targeted area. Each form is ergonomically designed to fit the natural curves and dimensions of the intended facial region.

The forms can be manufactured from beneficial gemstone materials that provide additional therapeutic properties for skin health and treatment. The contoured structure, combined with the ridges 201, creates a comprehensive lifting effect while the optional gemstone composition offers supplementary benefits for skin wellness.

This innovative design integrates three key elements: the anatomically-specific form shape, the mechanical lifting action of the contoured ridges 201, and the potential therapeutic properties of gemstone materials. Together, these create a comprehensive facial treatment device that can be customized for different areas of the face and individual skin needs.

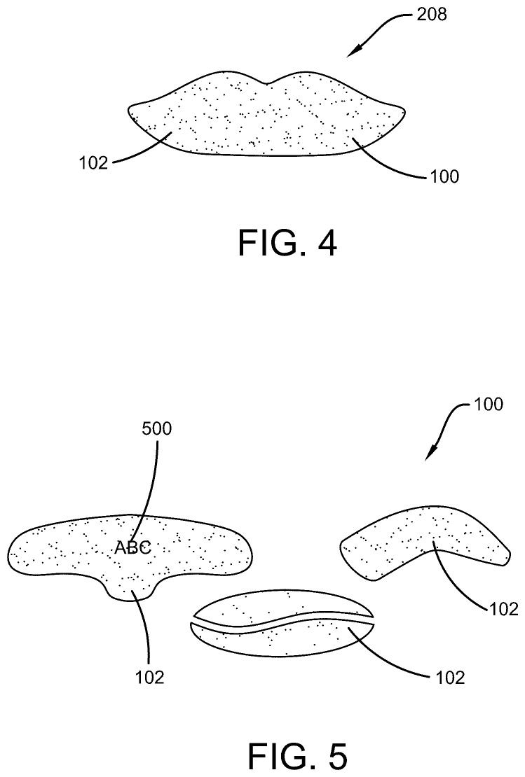

Specifically, as shown in FIGS. 2-4, in one embodiment, the body component 102 is configured in a curved, teardrop-like shape 200, which is sized and shaped to be positioned under a user's cheekbone, under a user's eye area and/or along the sides of a user's nose. In another embodiment, the body component 102 is configured in a curved, oblong oval shape 202 with a protruding bulbous area 204 extending from the middle edge 206 of the patch 102. This embodiment is sized and shaped to be positioned on a user's forehead and above a user's eyes, with the protruding bulbous area 204 being positioned in the flat area between a user's eyebrows. In another embodiment, the body component 102 is configured in a curved, oblong oval shape 208, which is sized and shaped to be positioned above or below a user's mouth and extends around the sides of a user's mouth. The scope of invention should not be limited to the various exemplary shapes and sizes. There can be many other possible shapes with different sizes that can be derived based on users need.

In one embodiment, the curved, teardrop-like shape 200 is approximately 2.54 cm in length and approximately 5.08 cm in width. In one embodiment, the curved, oblong oval shape 202 with a protruding bulbous area 204 is approximately 3.81 cm in length and approximately 6.35 cm in width and approximately 0.64 cm in width of the protruding bulbous area 204. In one embodiment, the curved, oblong oval shape 208 is approximately 6.35 cm in length and approximately 1.9 cm in width.

Further, the shape of the body component 102 is designed to not only fit comfortably on the designated area of the face, but to also taper at the edges to minimize creasing and pressure on the skin, as not to create additional wrinkles. Further, the body component 102 can be specially designed and custom matched to the facial contours of a user's forehead, under eye region, smile lines, dimple lines, and the sides of the mouth and nose, etc. Thus, the stone therapy device 100 ensures perfect fit for each facial zone, while preserving the natural properties of the gemstone 104. In use, the tiger's eye gemstone 104 can help revitalize skin, by diminishing wrinkles, reducing puffiness, and soothing sensitive skin, as well as can help fortify blood, alleviate sluggishness, and bring back feelings of motivation, etc.

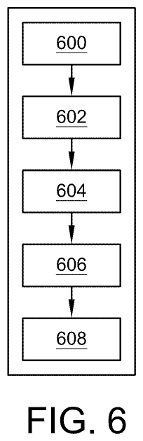

As shown in FIG. 5, although the stone therapy device 100 of the present invention is mainly developed for face, neck and décolleté areas (i.e., used as a face patch), it may also be used for application to other regions of the body as well.

In yet another embodiment, the stone therapy device comprises a plurality of indicia 500. The body component 102 of the device 100 may include advertising, a trademark, or other letters, designs, or characters, printed, painted, stamped, or integrated into the body component 102, or any other indicia 500 as is known in the art. Specifically, any suitable indicia 500 as is known in the art can be included, such as but not limited to, patterns, logos, emblems, images, symbols, designs, letters, words, characters, animals, advertisements, brands, etc., that may or may not be gemstone, face patch, or brand related.

FIG. 6 illustrates a flowchart of the method of rejuvenating the face with facial patches. The method includes the steps of at 600, providing a stone therapy device comprising a body component shaped in a face form. The method also comprises at 602, choosing a specifically sized body component which corresponds with a specific area of the face. Further, the method comprises at 604, removing the release liner from the adhesive backing of the body component. The method also comprises at 606, applying the body component to a specific area of the face. Finally, the method comprises at 608, removing the body component after a period of time and disposing it.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “stone therapy device”, “therapy device” and “device” are interchangeable and refer to the stone therapy device 100 of the present invention.

Notwithstanding the forgoing, the stone therapy device 100 of the present invention can be of any suitable size and configuration as is known in the art without affecting the overall concept of the invention, provided that it accomplishes the above stated objectives. One of ordinary skill in the art will appreciate that the stone therapy device 100 as shown in FIGS. 1-6 is for illustrative purposes only, and that many other sizes and shapes of the stone therapy device 100 are well within the scope of the present disclosure. Although the dimensions of the stone therapy device 100 are important design parameters for user convenience, the stone therapy device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A stone therapy device that provides a user with facial forms to deliver wellness benefits to a user's face, the stone therapy device comprising:
a body component;
wherein the body component is shaped in various facial forms; and
further wherein the body component delivers enhanced wellness and therapeutic benefits.

2. The stone therapy device of claim 1, wherein the body component is configured as a facial patch to be applied to various areas of a user's face, during use and comprises strategically positioned ridges that work to lift and support face skin when applied.

3. The stone therapy device of claim 2, wherein the body component comprises a top surface facing out from a user's face, a bottom surface that contacts a user's face, and a rounded side edge, which defines a shape of the body component.

4. The stone therapy device of claim 3, wherein the body component comprises an adhesive backing for adhering the body component to skin.

5. The stone therapy device of claim 4, wherein the adhesive backing has an adhesion value that allows for general ease of application and removal from skin, such as approximately 18 to 24 ounces/inch width.

6. The stone therapy device of claim 5, wherein the body component can remain on skin for as little or long as desired up to 24 hours at a time and can be easily removed from skin by pulling the body component across itself 180° and peeling it off for disposal.

7. The stone therapy device of claim 6, wherein the body component is preferably used one time, however, it can be used as many times as possible, until it loses its stickiness.

8. The stone therapy device of claim 7, wherein the body component comprises a release liner, which forms a backing for the adhesive backing and is removed for use.

9. The stone therapy device of claim 8, wherein the body component is entirely composed of genuine tiger's eye gemstone.

10. The stone therapy device of claim 9, wherein the body component utilizes piezoelectric technology to generate electric fields (EFs) based on pressure, which modulate skin cell behaviors.

11. The stone therapy device of claim 10, wherein the body component is configured in a curved, teardrop-like shape.

12. The stone therapy device of claim 10, wherein the body component is configured in a curved, oblong oval shape with a protruding bulbous area extending from a middle edge of the body component.

13. The stone therapy device of claim 10, wherein the body component is configured in a curved, oblong oval shape.

14. A stone therapy device that provides a user with facial forms to deliver wellness benefits to a user's face, the stone therapy device comprising:
a body component comprises a top surface facing out from a user's face, a bottom surface that contacts a user's face, and a rounded side edge, which defines a shape of the body component; and
wherein the body component is configured as a facial patch to be applied to various areas of a user's face, during use and comprises strategically positioned ridges that work to lift and support face skin when applied;
wherein the body component is shaped in various facial forms;
wherein the body component comprises an adhesive backing for adhering the body component to skin;
wherein the body component comprises a release liner, which forms a backing for the adhesive backing and is removed for use;
wherein the body component is entirely composed of genuine tiger's eye gemstone;
wherein the body component utilizes piezoelectric technology to generate electric fields (EFs) based on pressure, which modulate skin cell behaviors; and
further wherein the body component delivers enhanced wellness and therapeutic benefits.

15. The stone therapy device of claim 14, wherein the body component is configured in a curved, teardrop-like shape that is approximately 2.54 cm in length and approximately 5.08 cm in width.

16. The stone therapy device of claim 14, wherein the body component is configured in a curved, oblong oval shape with a protruding bulbous area extending from a middle edge of the body component that is approximately 3.81 cm in length and approximately 6.35 cm in width and approximately 0.64 cm in width of the protruding bulbous area.

17. The stone therapy device of claim 14, wherein the body component is configured in a curved, oblong oval shape that is approximately 6.35 cm in length and approximately 1.9 cm in width.

18. The stone therapy device of claim 14, wherein the body component can remain on skin for as little or long as desired up to 24 hours at a time and can be easily removed from skin by pulling the body component across itself 180° and peeling it off for disposal.

19. The stone therapy device of claim 14 further comprising a plurality of indicia.

20. A method of rejuvenating the face with facial patches, the method comprising the following steps:
providing a stone therapy device comprising a body component shaped in a face form;
choosing a specifically sized body component which corresponds with a specific area of the face;
removing the release liner from the adhesive backing of the body component;
applying the body component to a specific area of the face; and
removing the body component after a period of time and disposing it.
